(19) United States
(12) Patent Application Publication
(10) Pub. No.: US 2023/0240624 A1
(43) Pub. Date: Aug. 3, 2023

(54) SYSTEM FOR MONITORING VITAL SIGNS

(71) Applicant: Adam Benjamin Tannenbaum, Ashdod (IL)

(72) Inventor: Adam Benjamin Tannenbaum, Ashdod (IL)

(21) Appl. No.: 17/590,364

(22) Filed: Feb. 1, 2022

(51) Int. Cl. A61B 5/00; A61B 5/0205

(52) U.S. Cl. CPC A61B 5/746; A61B 5/02055; A61B 5/7278; A61B 5/7282; A61B 5/024

(57) ABSTRACT

The invention is a low-cost, easily installed, vital signs monitoring system that can provide enhanced monitoring and response for non-ICU patients.

### TECHNICAL FIELD

The present invention relates to means for monitoring a person's vital signs.

### BACKGROUND OF THE INVENTION

In intensive-care units (ICUs), patients are monitored constantly for a variety of vital signs comprising pulse rate, blood pressure, brain activity and the like. When a person is categorized as critical, it is essential to monitor on a continuous basis vital signs in order to respond quickly to any sudden changes. Such systems are typically interfaced to monitors located at nearby nurses' stations so that any alarm condition can be responded to very quickly.

In times of widespread epidemic, hospitals and other patient-care facilities can quickly become overwhelmed with patients which can markedly affect the ratio of patients-to-care givers to a point where response times can be severely compromised. Thus, even though many recent patients may not yet be critical, it may be difficult to impossible to monitor their vital signs frequently enough to provide timely response to changes.

As became very apparent in the Covid pandemic starting in 2020, ICU beds and monitoring equipment can become quickly occupied by a large influx of patients creating triage situations and forcing medical personnel to make do with fewer people, beds and equipment. As a result, clearly some patients who are not yet critical may be assigned to temporary beds in corridors lacking any monitoring equipment and subjecting them to much higher risk of delayed response and more serious consequences including death.

An ICU with its specialized monitoring equipment is very costly, and ICU size is typically dictated by assessments of patient loading. In a pandemic, or local catastrophic situation, loading can quickly exceed worst-case anticipation. The end result is a higher number of deaths.

If it were possible to have easily deployable monitoring systems that could adequately monitor patient vitals, it could enable medical personnel to more quickly respond to sudden change situations that might be overlooked without more frequent monitoring. In many cases people who are sent home for care, to rehab facilities or long-term care homes, each need a way to be monitored continuously.

### BRIEF SUMMARY OF THE INVENTION

The system herein disclosed and claimed is a monitoring system that can be quickly deployed and provide monitoring of such vitals as pulse rate, respiration, oxygen saturation, and body positioning providing continuous monitoring with real-time alarm triggering that is reported, wirelessly, to nearby servers/monitoring stations as well as via the Internet to a centralized oversight system.

The system uses readily available sensors for image, sub-audible vibration, heat, sound, oxygen saturation, and air quality molecule and particulate detection to monitor a person's respiration and any breathing difficulties, body movements such as spasms or choking or seizures, release of bodily fluids or solids, and signs of distress and calls for help.

The sensors interface with a microcontroller programmed to rapidly scan sensor inputs and do algorithmic processing to quickly determine any significant deviation from normal readings. The sensors and microcontroller are located in a non-conductive enclosure, similar in size and shape to a smoke detector, in which there is a self-contained power supply (e.g. a battery and power bus) and a wireless transceiver that sends and receives signals from a local or remote server using a standard wireless protocol, such as Wi-Fi, Cellular or Bluetooth, but where all signals sent are encrypted so that HIPAA regulations are adhered to and privacy is preserved.

One area of novelty is the enclosure and its multipurpose attachment fixture. It may be attached to a wall, a bed's side or foot rail, a wheelchair's arm structure, a walker and the like. The attachment fixture is designed to allow the unit and its sensor apertures to be positioned, automatically, so as to provide inputs from only one person despite the possibility of being in a room with other patients, creating, in effect, a single-patient monitoring zone. When a patient moves, or is moved, to a new position, the monitoring system will detect the change and attempt to automatically establish a monitoring zone for the new position. If the system cannot establish a new monitoring zone for the patient, it will invoke an alarm. The attachment fixture also makes use of common wall-hook anchors, magnetic coupling to metal rails, and so on. The system offers a Bluetooth connection to multiple remote devices that the person might wear for data capture, like a wrist band, a ring, or pad in the wheelchair.

Low cost is another novel aspect in that the sensors, microcontroller, power source, and wireless transceiver are all available from multiple sources at commodity-level pricing. These systems can be made in large quantities, stored until needed, rapidly deployed and set up. Their deployment and use can create a level of accommodation that is less stringent and costly than an ICU bed and monitoring equipment while providing monitoring capability far above that of a typical hospital room or long-term care facility bed.

### DESCRIPTIONS OF THE DRAWINGS

FIG. 1 depicts one embodiment of the system.

FIG. 2 depicts a second embodiment of the system.

FIG. 3 depicts system inside an enclosure.

FIG. 4 depicts system mounted to a wall.

FIG. 5 depicts system mounted to a patient bed.

FIG. 6 depicts system mounted to a wheelchair's arm structure.

### DETAILED DESCRIPTION OF THE INVENTION

In the wake of the global pandemic that began in 2020, virtually every large country found its healthcare systems overwhelmed with an influx of patients and overburdened ICUs and medical staff. Using de facto triage decision making, medical personnel were putting the most serious Covid cases in ICUs and others in makeshift beds often located in hallways. It was typically the case that too few medical personnel were monitoring too many patients at any time.

Patients in ICU rooms had the benefit of advanced monitoring systems that surveilled their vital signs continuously. Even so, alarm situations were not attended to as quickly as warranted because of too many patients and too few medical staff.

Patients located in non-ICU areas or long-term care facilities were monitored as often as could be done by overburdened medical staff shuttling quickly from bed to bed. More often there were no monitoring systems used to keep a check on a non-ICU patient's vitals. It was often a case of serendipity if a sudden change in vitals was caught early. Too often, changes in vitals were discovered too late to avoid consequential complications.

ICU beds and monitoring equipment are costly compared to non-ICU facilities. As a result, hospital management is forced to build to accommodate average anticipated patient loading. In the Covid pandemic, it was typically the case that all ICU units were filled and even patients needing ICU-type care were put in hallways crowded with beds to await an ICU bed vacancy. Many such patients expired before they could be moved. Other patients with less serious symptoms upon entry had to depend upon overburdened medical staff to be monitored and treated. Many such patients later developed more serious symptoms that went undiscovered until consequential organ damage had occurred. There was no monitoring of the air quality for pathogens, from regular occupancy to overcrowded occupancy.

The system herein disclosed and claimed is a monitoring system that can be quickly deployed and provide monitoring of such vitals as pulse rate, respiration, and oxygen saturation, providing continuous monitoring with real-time alarm triggering that is reported, wirelessly, to nearby servers/monitoring stations and could as well be reported via the Internet to a centralized oversight system; a designated caregiver, or a remotely located family member.

The system comprises a plurality of electronic sensors for detecting images, sub-audible vibration, heat, sounds, oxygen saturation levels, and molecules/particulates in the air. With those sensor inputs, a microcontroller equipped with one or more programs designed to algorithmically compare sensor data to appropriate models can determine in near real-time whether a change in data indicates a change in patient condition that merits an alarm condition. In the event of an alarm, routine sensor-date processing is interrupted and an alarm condition is triggered followed by sensor-data processing.

While receiving sensor data, the microcontroller continuously sends, via a wireless transceiver, sensor-data updates to a server located within reliable wireless communications range or in remote situations by cellular communications. Before sending such data, the system establishes a link with the server wherein it is associated with a specific patient during set up. The system also previously establishes a single-patient monitoring zone. Thus, the microcontroller's sensor-data updates are associated with that patient although the wireless data transmissions are encrypted before being transmitted in order to protect patient privacy and to conform to HIPAA regulations. Upon receipt by the server, the encrypted data is rapidly decrypted and sensor-data results displayed. An alarm condition causes the server and monitoring system to emit attention-getting audible signals, such as an on-off beeping sound or bell sound

The server may also be connected via the Internet to a centralized oversight system that monitors and stores all the data received from multiple monitoring systems. Given the gravity of the data. the monitored person's actual personal ID data may not be saved on the system server. Instead, it may be encrypted in a block-chain server system, thus adding an extra level of security.

The portion of the system comprising the sensors, microcontroller and wireless transceiver is contained within a non-conducting enclosure similar in size and shape to a typical smoke detector. It also contains a power source, such as a battery, and a power bus, which provides power to the sensor, microcontroller and wireless transceiver subsystems. It is this enclosed system portion that is located in proximity to the patient to be monitored. An oxygen-saturation sensor and other vitals sensors may be located on the patient's finger or wrist and wirelessly linked to the monitoring system.

During monitor system set up, the system should be located such that the sensors have a relatively clear path to the patient. For example, the image sensor should be focused on the patient along with the sound sensor. Apertures in the enclosure plus a small, low-power fan pulling in air allows air, light and sound to enter the sensors contained within the enclosure.

The monitoring system enclosure is designed such that on the back face (e.g. the side opposite the enclosure apertures) there is a ball firmly attached to it which fits snuggly inside a mounting fixture's ball joint. The interface is very similar to that used to attach an automobile's rear-view mirror to its windshield mounting fixture. It provides a firm attachment that allows the attached monitoring system to be adjusted in the vertical and horizontal planes so that it can be positioned to provide the direct path between its apertures and the patient. A motorized positioning device contained within the mounting fixture, and interfaced to the microcontroller via a conductive or wireless data path, is operative to automatically position the monitoring system such that a single-patient monitoring zone is established. If a patient's position is changed, the microncontroller and motorized positioner execute a closed-loop algorithm to re-establish a single-patient monitoring zone. If that zone cannot be re-established, the monitoring system will invoke an alarm.

The mounting fixture is operative to mount it to a vertical wall using a suitable wall hook such as that which is used to hang a picture frame. It is also operative to magnetically bond with a metal surface such as the side or foot rail at the side or end of a patient's bed, or the arm-rest structure on a wheelchair, or a walker. These are just a few exemplary ways in which the monitoring subsystem may be mounted.

The sensors may be discrete components or combined in modular form factors. The microcontroller comprises a central-processing unit (CPU), input/output interfacing, program memory, data memory, and counters.

The system enclosure may be made of plastic or resinous material and should not block or significantly attenuate incoming or outgoing wireless wave energy.

Power is provided by a self-contained battery which may or may not be rechargeable. In the case where the battery is rechargeable, there may be an interface for connecting the monitoring system to a charger. In any case, the enclosure should allow easy replacement of a battery.

One or more programs residing in the microcontroller are operative to process incoming sensor data that has been received and converted into appropriate digital format. The one or more programs using algorithmic processing quickly compare incoming sensor data to stored data models. If the incoming data is consistent with normal data model range, the results are stored and concurrently formatted into appropriate encrypted form for wireless transfer to a wirelessly-linked server. If the sensor data falls outside the normal data model range, it will trigger an interrupt alarm that takes precedence over any queued sensor-data messages and initiates an alarm condition. In this way, non-ICU patients can be monitored continuously and responded to upon any alarm condition. One immediate benefit is that a single medical person can quickly respond to a need situation despite having many patients being monitored. In the absence of an alarm condition, medical staff can perform routine monitoring checks. An alarm condition takes precedence over routine and requires quick response, of course.

With regard to monitoring breathing and respiration rate, the monitoring system uses three collaborating sensors—image, sub-audible vibration and sound. Focused on a patient, and more particularly, a rising and falling chest, the image sensor data, the vibration sensor data and sound sensor data are synchronized so that sounds and vibration related to breathing correspond to inhalation and exhalation chest motion. In this way, the three sensors ensure that the data corresponds to the correct patient's breathing, and establishes a single-patient monitoring zone.

Clearly, as a patient shifts from, say, sitting in a wheelchair to lying on or being placed in a bed, the monitoring system apertures need to have their positions adjusted. This adjustment is done automatically by the microcontroller in conjunction with the mounting fixture's positioning motor. While monitoring vitals, the system may monitor the patient's body position, so that an alarm notification is triggered if the needed rotation/movement is not detected. Additionally, while monitoring vitals, in the cases of regularly needed treatment, the monitoring system will trigger an alarm, based on users' data, if the treatment was not done, such as dialysis, for example. While monitoring vitals, the system can monitor the air quality in a room to report and/or trigger an alarm where unsafe levels of, say, carbon monoxide or carbon dioxide are detected.

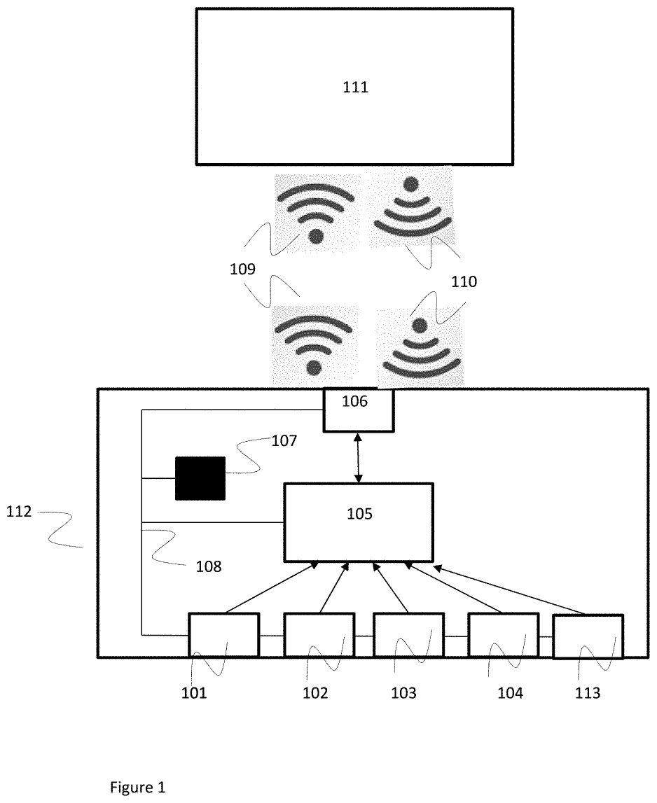

Looking at the drawings and figures can provide further elucidation about the structure and function of the monitoring system In FIG. 1, an image sensor (101), sound sensor (102), air-quality sensor (103), sub-audible vibration sensor (104) and infrared heat sensor (113) all interface conductively with the microcontroller (105) as shown. A battery power source (107) makes use of a power bus (108) to provide power to sensors, microcontroller and wireless transceiver (106). These subsystems are located inside an enclosure (112). Encrypted sensor data is converted to wireless signals (109) which are transmitted into space and received by a server (111) having a compatible wireless transceiver. Control signals (110) sent from the server to the enclosed sensors-microcontroller-wireless transceiver subsystem are received and conveyed to the microcontroller for processing and action.

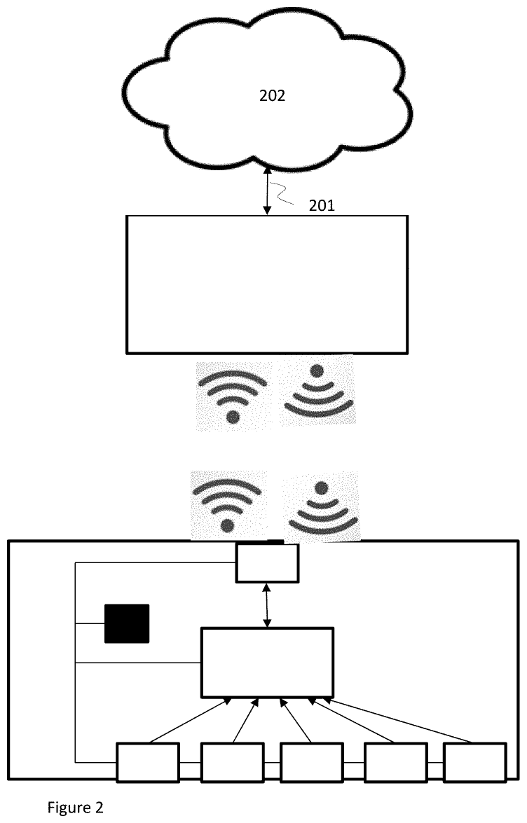

FIG. 2 shows the system embodiment of FIG. 1 expanded to include a signal interface (201) providing connection to the Internet via the cloud (202). This would enable each monitoring system to become part of larger organization of monitors including remote data capture and storage.

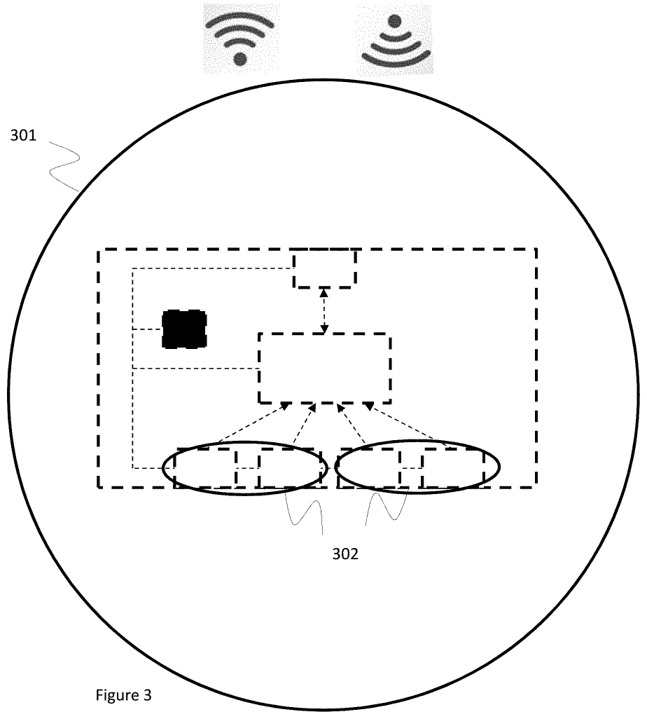

In FIG. 3 the embodiment of the sensors-microcontroller-wireless transceiver subsystem is contained within an enclosure (301) wherein one or more apertures (302) allow light, sound and air to impinge upon the sensors.

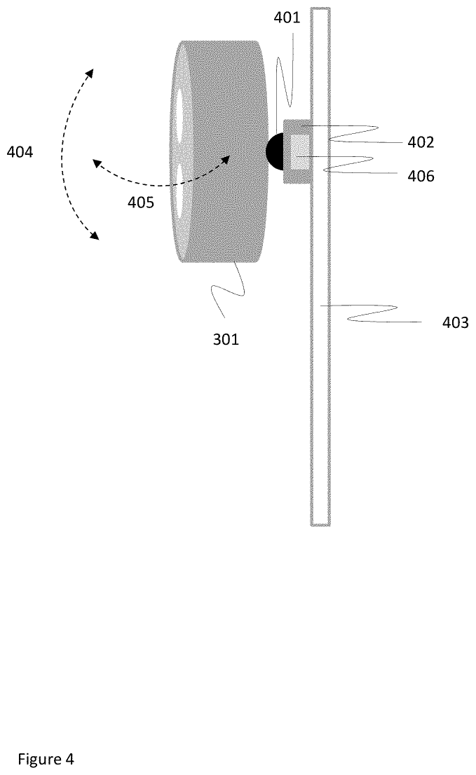

In FIG. 4, the sensors-microcontroller-wireless transceiver subsystem's enclosure (301) is shown mounted to a vertical wall (403). The attachment fixture (402) comprises a ball joint which snugly accommodates a ball (401) firmly attached to the enclosure (301). The attachment fixture enables positioning of the enclosure in horizontal (405) and vertical (404) planes by using its positioning motor in conjunction with microcontroller coordination. Once adjusted, the snug ball-joint fit ensures that the enclosure remains in the new position.

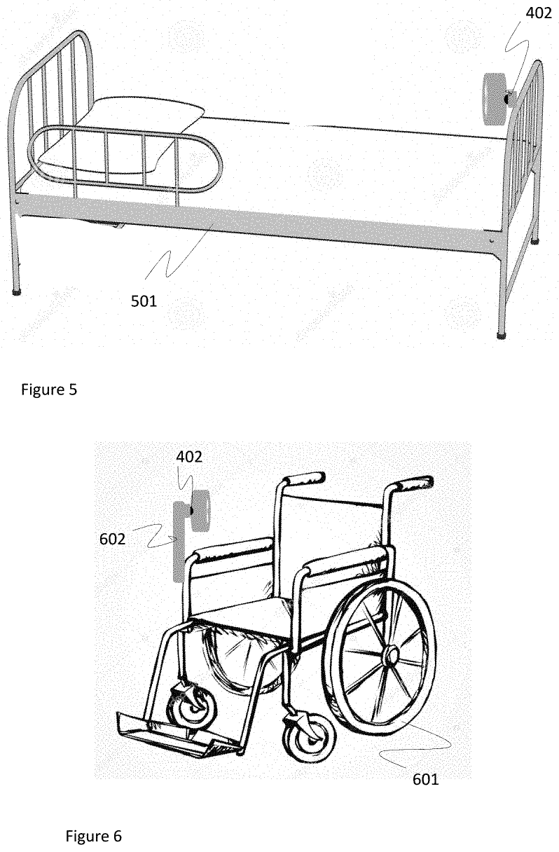

The wall-mounted monitoring system is just one of many placement and mounting options. As shown in FIG. 5, the attachment fixture (402) may be outfitted with a strong magnet such that it can be firmly mounted to a metal foot rail of a hospital bed (501).

FIG. 6 shows another mounting option where a metal tube (602) is attached to the vertical portion of a wheelchair's (601) arm rest. The monitoring system's attachment fixture (402) equipped with a strong magnet or clamp may be mounted to the metal tube.

The figures are all exemplary and should not be seen as limiting the structure, function and scope of the invention.

What is claimed is:

1. A system comprising:
an image sensor operative to sense light and images;
a sub-audible vibration sensor operative to sense sub-audible vibration;
a sound sensor operative to sense audible sounds;
an infrared heat sensor operative to sense body heat levels;
an air-quality sensor operative to detect the presence of particulates and molecules in air;
said image, sub-audible vibration, sound, infrared heat and air-quality sensors interface with a microcontroller;
said microcontroller comprises a central processing unit, program memory, data memory, input-output interface and counters;
said microcontroller is operative to execute at least one program wherein sensor data received from said image, sub-audible vibration, sound, infrared heat and air-quality sensors are processed to determine if respiration, pulse rate, oxygen saturation and temperature vital signs are normal or abnormal;
said microcontroller is operative to trigger an alarm condition if any said respiration, pulse rate, oxygen saturation and temperature vital signs are determined to be abnormal;
a wireless transceiver operative to send and receive wireless signals;
said wireless signals sent by said wireless transceiver are encrypted by said microcontroller before being conveyed from said microcontroller to said wireless transceiver for sending;
a battery operative to provide a power source;
a power bus operative to distribute power from said battery to said image, sub-audible vibration, sound, infrared heat and air-quality sensors; to said microcontroller; and to said wireless transceiver;
an enclosure containing said image, sub-audible vibration, sound, infrared heat and air-quality sensors; said battery; said power bus; said microcontroller; and said wireless transceiver;
said enclosure is non-metallic and non-conductive;
said enclosure has at least one aperture on a surface in direct line with at least one sensor;
said enclosure has a solid ball attached to its outer surface on side opposite said at least one aperture;
said solid ball is operative to fit in a ball joint of an attachment fixture;
said attachment fixture is operative to be mounted firmly to a wall surface or metallic structure;
said solid ball is operative to be rotated with respect to said attachment fixture by a motorized positioning device residing within said attachment fixture;
said motorized positioning device is operative to respond to control signals from said microcontroller;
said motorized positioning device is operative to convey position signals to said microcontroller;
said enclosure and all components contained therein comprises a monitoring subsystem;
said wireless transceiver is operative to send wireless signals to a server, and to receive wireless signals from said server; and
said monitoring subsystem and said server comprises a monitoring system.

2. A claim as in claim 1 further comprising:
said monitoring system receives control wireless messages conveyed via said server and emanating from a central system wherein said control wireless messages are conveyed via the Internet.; and
said monitoring system sends encrypted messages to said server which are in turn conveyed via the Internet to said central system wherein such messages are decrypted and stored.
